(19) United States
(12) Patent Application Publication
(10) Pub. No.: US 2011/0144211 A1
(43) Pub. Date: Jun. 16, 2011

(54) USE OF MICROCRYSTALLINE CELLULOSE FOR INTERFERING WITH THE EXTRACTION OF EPHEDRINE

(76) Inventors: Lars Hermann, Schindellegi (CH); Marcel Becker, Au (CH)

(21) Appl. No.: 12/635,888

(22) Filed: Dec. 11, 2009

(51) Int. Cl. A61K 31/137; A61P 11/00

(52) U.S. Cl. 514/653

(57) ABSTRACT

Use of microcrystalline cellulose and, optionally, a surfactant in an aqueous pharmaceutical composition in order to inhibit the extraction of ephedrine for the purpose of drug abuse.

The present invention refers to the use of microcrystalline cellulose and, optionally, a surfactant in an aqueous pharmaceutical composition in order to inhibit the extraction of ephedrine for the purpose of drug abuse.

### BACKGROUND OF THE INVENTION

Ephedrine is a sympathomimetic drug used in the treatment of cough, rhinitis, hay fever and asthma bronchiale. Due to its stimulating and appetite suppressing effect ephedrine has a high potential of being abused. Moreover, ephedrine can serve as a precursor in the synthesis of other amphetamines, in particular N-methylamphetamine (“Crystal” or “Meth”).

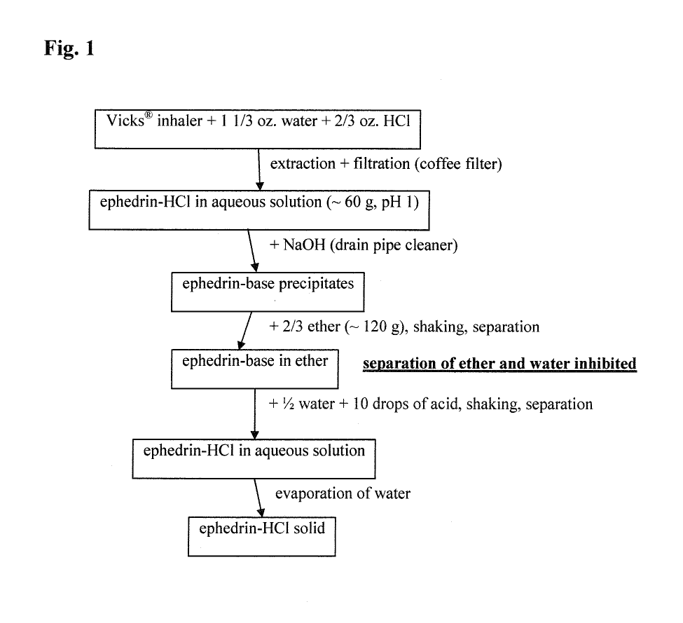

For the purpose of abuse ephedrine is extracted from pharmaceutical products, e.g. Vicks® nasal inhaler, commercially available in the US at drug stores. Instructions on how to recover ephedrine can be found on the interne (see, for example, http://www.phrack.org). Accordingly, the inner package of the pharmaceutical product is destroyed and both the package material and pharmaceutical composition is treated with a strong hydrochloric solution (see FIG. 1). Such a solution is obtained by diluting a brick and driveway cleaner purchased at the hardware store in water. The resulting solution is filtered through a commonly available coffee filter. Addition of solid sodium hydroxide contained in drain cleaners produces an alkaline shift in pH leading to a precipitation of free ephedrine base [1, 2]. Then, this solution is mixed with diethyl ether at a volume ratio of one-third aqueous solution and two-third organic solvent. Since the solubility of free ephedrine base in diethyl ether is superior over its water solubility the drug will enter into the organic solvent [1, 2, 3]. Thus, after partition of the aqueous and organic phases, the aqueous layer is discarded while the organic layer is used for further processing.

Due to its great volatility [1, 2] free ephedrine base is converted into the less volatile hydrochloride salt by mixing the organic phase with an equal volume of water slightly acidified by hydrochloric acid. In contrast to the free base, ephedrine hydrochloride is virtually insoluble in diethyl ether but dissolves well in water [1, 2]. Thus, after partition of the aqueous and organic phases, the organic layer is discarded and ephedrine hydrochloride is obtained after evaporation of the aqueous solvent at room temperature.

There is a need to make the abusive recovery of ephedrine from commercially available pharmaceutical products much more difficult. Thus, the object of the present invention is to provide methods and means interfering with ephedrine extraction.

### SUMMARY OF THE INVENTION

The object of the present invention is solved by a method for inhibiting a phase separation in a mixture of an aqueous pharmaceutical composition comprising ephedrine or a salt or a derivative thereof and one or more water-insoluble organic solvents, preferably a diethyl ether, the method comprising the step of adding one or more microcrystalline celluloses to the aqueous pharmaceutical composition.

In one embodiment, the method further comprises the step of adding one or more surfactants to the aqueous pharmaceutical composition.

The object of the present invention is further solved by a method of using one or more microcrystalline celluloses for inhibiting an extraction of ephedrine or a salt or a derivative thereof from an aqueous pharmaceutical composition.

In one embodiment, the method further uses one or more surfactants.

The object of the present invention is further solved by an aqueous pharmaceutical composition comprising ephedrine or a salt or a derivative thereof and one or more microcrystalline celluloses.

In one embodiment, the aqueous pharmaceutical composition further comprises one or more surfactants.

In one embodiment of the methods or the aqueous pharmaceutical composition, the aqueous pharmaceutical composition is selected from the group comprising a nasal spray composition, an asthma spray composition and a cough syrup. A nasal spray composition is particularly preferred.

In one embodiment of the methods or the aqueous pharmaceutical composition, the microcrystalline cellulose is present in the aqueous pharmaceutical composition at an amount of about 1.0% to about 10.0% by weight, preferably of about 1.4% to about 8.7% by weight, and most preferably at about 1.4%, 3.0%, 4.7%, 5.0%, 6.3%, 8.6%, or 8.7% by weight.

In one embodiment of the methods or the aqueous pharmaceutical composition, the microcrystalline cellulose has a nominal particle size of about 10 μm to about 50 μm, preferably of about 15 μm to about 25 μm, and most preferably of about 15 μm, 20 μm or 25 μm.

In one embodiment of the methods or the aqueous pharmaceutical composition, the microcrystalline cellulose is selected from the group comprising Avicel® PH105, VivaPur® 105 and Emcocel® SP15.

In one embodiment of the methods or the aqueous pharmaceutical composition, the surfactant is present in the aqueous pharmaceutical composition at an amount of about 1.0% to about 5.0% by weight, preferably of about 1.4% to about 3.9% by weight, most preferably of about 1.4%, 2.9%, 3.0%, 3.3% or 3.9% by weight.

In one embodiment of the methods or the aqueous pharmaceutical composition, the surfactant is a polysorbate, preferably is Tween® 20 or Tween® 80.

The present invention provides a method for inhibiting, i.e. preventing or reducing, a phase separation in a mixture of an aqueous pharmaceutical composition comprising ephedrine and diethyl ether used for extracting the drug. The effect of such inhibition is that a recovery of ephedrine from the aqueous pharmaceutical composition becomes much more difficult and laborious, i.e. impractical.

According to the present invention, the mixture (emulsion) of the aqueous solution and the organic solvent is stabilized by adding to the aqueous pharmaceutical composition (1) microcrystalline cellulose (MCC) and, optionally, (2) a surfactant (or mixtures of surfactants). In order to be effective with regard to a prevention of drug abuse, microcrystalline cellulose and the optional surfactant must be included in the original aqueous pharmaceutical composition as it is commercially available.

Microcrystalline cellulose is widely used as an additive in food and pharmaceutical industry. As a white, free flowing powder it is available with different particle sizes. In pharmaceutical tablets, in particular in tablets produced by direct tabletting, microcrystalline cellulose is commonly used as a vehicle and disintegrating agent [4]. In food technology microcrystalline cellulose is used as a thickening agent since it has the capability of forming stable gels in aqueous media [5]. The viscosity of such gels increases with increasing solid contents of microcrystalline cellulose, and solids contents of 10% and above are employed.

The present invention is based on the finding that the addition of microcrystalline cellulose to an aqueous pharmaceutical composition significantly delays the phase separation after mixing the same with diethyl ether. Obviously, microcrystalline cellulose stabilizes the emulsion of diethyl ether and water. Only a very small volume of clear organic solvent separates from the emulsion after a while, and this small organic layer is difficult to remove and does not contain considerable amounts of drug.

In one aspect of the present invention, the solids content of microcrystalline cellulose is of importance since this affects the viscosity of the emulsion of diethyl ether and the aqueous pharmaceutical composition. The higher the viscosity of the emulsion the lesser is the chance that individual water droplets or individual diethyl ether droplets converge and combine. On the other hand, it may be desirable to limit the viscosity of the original aqueous pharmaceutical composition. While the viscosity may be less relevant in case of e.g. a cough syrup, it may matter in case of an aqueous pharmaceutical composition intended for use with a spraying, nebulizing or vaporizing device, e.g. a nasal spray or asthma spray.

In another aspect of the present invention the particle size of microcrystalline cellulose is of particular importance. On the one hand, the particle size affects the extent to which the emulsion is stabilized. It is assumed that, just as for the formation of gels, the microcrystalline cellulose particles form a kind of stable mesh structure trapping both water and diethyl ether droplets. Thus, individual water or diethyl ether droplets are immobilized and their converging and combining is inhibited. On the other hand, in order that the invention works it is essential that the microcrystalline cellulose particles are not retained by the coffee filter which is used prior to the addition of diethyl ether to the aqueous pharmaceutical composition.

Another finding of which the present invention takes advantage is that the addition of a surfactant to the aqueous pharmaceutical composition further supports the inhibition of the phase separation. A surfactant reduces the interface tension between the aqueous and organic phases and thus assists in the stabilization of the emulsion. The surfactant should be water-soluble and should not adversely affect the action of the original aqueous pharmaceutical composition.

### DETAILED DESCRIPTION OF THE INVENTION

In the following, the present invention will be described in more detail by means of the accompanying figures and the examples.

FIG. 1 is a flow chart demonstrating the steps involved in the abusive extraction of ephedrine from a Vicks® nasal inhaler and where in this process the present invention interferes with.

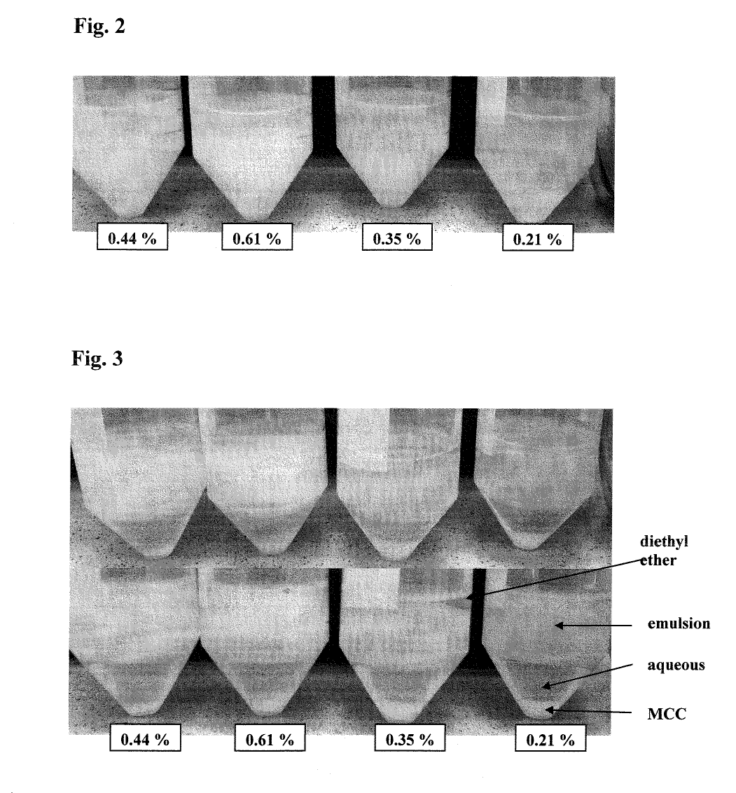

FIG. 2 is a photography showing an aqueous pharmaceutical composition containing microcrystalline cellulose directly after mixing with diethyl ether. Percentages of Avicel® PH105 in the mixtures: 0.44%, 0.61%, 0.35%, and 0.21% by weight.

FIG. 3 is a photography showing the mixtures as in FIG. 2 after being allowed to rest over night.

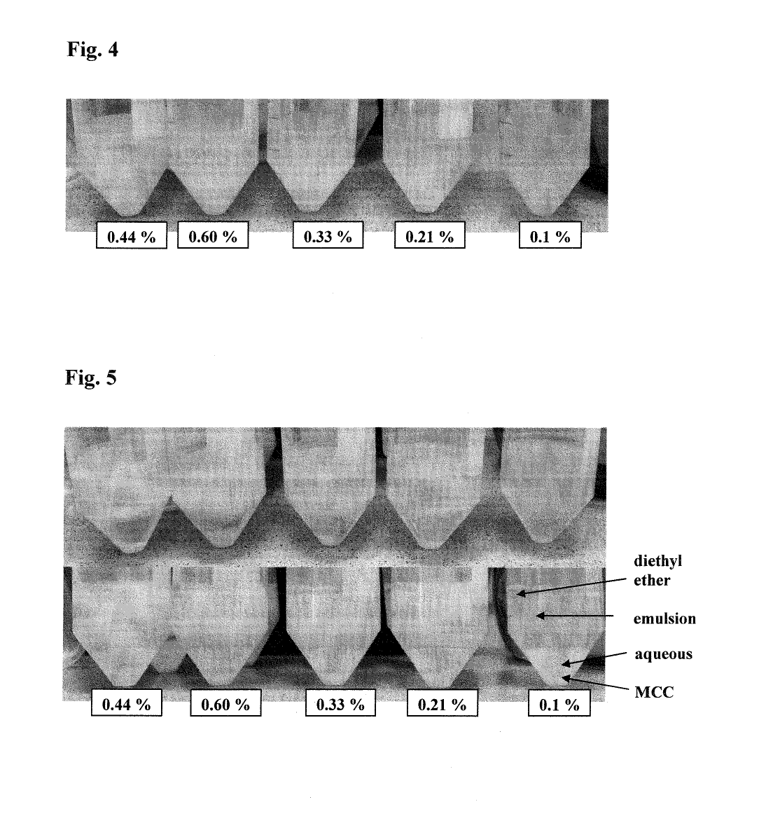

FIG. 4 is a photography showing an aqueous pharmaceutical composition containing microcrystalline cellulose and a surfactant of (Tween® 20) directly after mixing with diethyl ether. Percentages of Avicel® PH105 in the mixtures: 0.44%, 0.60%, 0.33%, 0.21%, and 0.1% by weight.

FIG. 5 is a photography showing the mixtures as in FIG. 4 after being allowed to rest over night.

### EXAMPLES

### Example 1

### Microcrystalline Cellulose

To the aqueous pharmaceutical composition of Vicks® nasal inhaler a microcrystalline cellulose, i.e. Avicel® PH105 (FMC Corporation, USA), nominal particle size 20 μm, was added at different percentages. Addition of sodium hydroxide resulted in pH>12. Then, diethyl ether was added and the mixtures were vigorously agitated (vortexed). The composition of the mixtures is shown in Table 1 below.

TABLE 1

| | Avicel ® | | Diethyl |
|---|---|---|---|
| No. | PH105 [g] | H₂O + NaOH [g] | Ether [g] |
| 1 | 0.0405 | 3.1492 | 6.0172 |
| | 0.44% (=6.3%) | | |
| 2 | 0.0553 | 3.0406 | 6.0095 |
| | 0.61% (=8.7%) | | |
| 3 | 0.0318 | 3.0209 | 6.0103 |
| | 0.35% (=5.0%) | | |
| 4 | 0.0199 | 3.4689 | 6.0153 |
| | 0.21% (=3.0%) | | |

In round brackets: % of aqueous pharmaceutical composition

FIG. 2 shows for all percentages of microcrystalline cellulose optically homogenous emulsions directly after mixing. A first phase separation could be observed after about 30 minutes, i.e. a separation between an aqueous and an emulsion layer (not shown). As shown in FIG. 3, the emulsion layers are still maintained after the mixtures were allowed to rest over night with only a very small volume of diethyl ether being separated.

It also turned out that the high pH does not affect the stabilization of the emulsion.

Compared to Avicel® PH105, 0.2%, other microcrystalline celluloses, i.e. Emcocel SP15, nominal particle size 15 μm, 0.2%, or VivaPur® 105, nominal particle size 25 μm, 0.2%, (both from J. Rettenmaier & Söhne GmbH & Co. KG, Rosenberg, Germany) did not produce any difference with regard to the capability of stabilizing the emulsion.

### Example 2

### Microcrystalline Cellulose and Surfactant

The pharmaceutical composition of a Vicks® nasal inhaler was similarly treated as described in Example 1 above except that Tween® 20 was additionally added. The composition of the mixtures is shown in Table 2 below.

TABLE 2

| | Avicel ® | | | Diethyl |
|---|---|---|---|---|
| No. | PH105 [g] | Tween ® 20 [g] | H₂O + NaOH [g] | Ether [g] |
| 5 | 0.0402 | 0.0194 | 3.0225 | 6.1145 |
| | 0.44% (=6.3%) | 0.21% (=3.0%) | | |
| 6 | 0.0546 | 0.0187 | 3.0488 | 6.0491 |
| | 0.60% (=8.6%) | 0.20% (=2.9%) | | |
| 7 | 0.0305 | 0.0187 | 3.0581 | 6.0281 |
| | 0.33% (=4.7%) | 0.23% (=3.3%) | | |
| 8 | 0.0198 | 0.0274 | 3.0883 | 6.0981 |
| | 0.21% (=3.0%) | 0.3% (=3.9%) | | |
| 9 | 0.0088 | 0.0096 | 3.0200 | 6.0448 |
| | 0.01% (=1.4%) | 0.10% (=1.4%) | | |

In round brackets: % of aqueous pharmaceutical composition

FIG. 4 shows for all percentages of microcrystalline cellulose optically homogenous emulsions directly after mixing. After about 5 hours, a phase separation could be observed, i.e. a separation between an aqueous and an emulsion layer without a clear organic phase being formed (not shown). As shown in FIG. 5, the emulsion layers are still maintained after the mixtures were allowed to rest over night with only a very small volume of diethyl ether being separated.

### REFERENCES

[1] EuAB 6.5, Monography Ephedrin, Ephedrin-HCl
[2] Comments on Monography Ephedrin in EuAB 6.5
[3] Lin, H. et al., J. Pharm. Sci. 82 (10), 1993, pp 1018-1026
[4] Reier, G. et al., J. Pharm. Sci 55 (5), 1966, pp 510-514
[5] Battista, O. A. et al., Ind. Eng. Chem. 54 (9), 1962, pp 20-29

1. A method for inhibiting a phase separation in a mixture of an aqueous pharmaceutical composition comprising ephedrine or a salt or a derivative thereof and one or more water-insoluble organic solvents, preferably a diethyl ether, the method comprising the step of adding one or more microcrystalline celluloses to the aqueous pharmaceutical composition.

2. The method according to claim 1, further comprising the step of adding one or more surfactants to the aqueous pharmaceutical composition.

3. The method according to claim 1, wherein the aqueous pharmaceutical composition is selected from the group comprising a nasal spray composition, an asthma spray composition and a cough syrup.

4. The method according to claim 1, wherein the microcrystalline cellulose is present in the aqueous pharmaceutical composition at an amount of about 1.0% to about 10.0% by weight, preferably of about 1.4% to about 8.7% by weight, and most preferably at about 1.4%, 3.0%, 4.7%, 5.0%, 6.3%, 8.6%, or 8.7% by weight.

5. The method according to claim 1, wherein the microcrystalline cellulose has a nominal particle size of about 10 μm to about 50 μm, preferably of about 15 μm to about 25 μm, and most preferably of about 15 μm, 20 μm or 25 μm.

6. The method according to claim 1, wherein the microcrystalline cellulose is selected from the group comprising Avicel® PH105, VivaPur® VivaPur® 105 and Emcocel® SP15.

7. The method according to claim 2, wherein the surfactant is present in the aqueous pharmaceutical composition at an amount of about 1.0% to about 5.0% by weight, preferably of about 1.4% to about 3.9% by weight, most preferably of about 1.4%, 2.9%, 3.0%, 3.3% or 3.9% by weight.

8. The method according to claim 7, wherein the surfactant is a polysorbate, preferably is Tween® 20 or Tween® 80.

9. A method of using one or more microcrystalline celluloses for inhibiting an extraction of ephedrine or a salt or a derivative thereof from an aqueous pharmaceutical composition.

10. The method according to claim 9, further using one or more surfactants.

11. The method according to claim 9, wherein the aqueous pharmaceutical composition is selected from the group comprising a nasal spray composition, an asthma spray composition and a cough syrup.

12. The method according to claim 9, wherein the microcrystalline cellulose is present in the aqueous pharmaceutical composition at an amount of about 1.0% to about 10.0% by weight, preferably of about 1.4% to about 8.7% by weight, and most preferably at about 1.4%, 3.0%, 4.7%, 5.0%, 6.3%, 8.6%, or 8.7% by weight.

13. The method according to claim 9, wherein the microcrystalline cellulose has a nominal particle size of about 10 μm to about 50 μm, preferably of about 15 μm to about 25 μm, and most preferably of about 15 μm, 20 μm or 25 μm

14. The method according to claim 9, wherein the microcrystalline cellulose is selected from the group comprising Avicel® PH105, VivaPur® 105 and Emcocel® SP15.

15. The method according to claim 10, wherein the surfactant is present in the aqueous pharmaceutical composition at an amount of about 1.0% to about 5.0% by weight, preferably of about 1.4% to about 3.9% by weight, most preferably of about 1.4%, 2.9%, 3.0%, 3.3% or 3.9% by weight.

16. The method according to claim 10, wherein the surfactant is a polysorbate, preferably is Tween® 20 or Tween® 80.

17. An aqueous pharmaceutical composition comprising ephedrine or a salt or a derivative thereof and one or more microcrystalline celluloses.

18. The aqueous pharmaceutical composition according to claim 17, further comprising one or more surfactants.

19. The aqueous pharmaceutical composition according to claim 17, wherein the aqueous pharmaceutical composition is selected from the group comprising a nasal spray composition, an asthma spray composition and a cough syrup.

20. The aqueous pharmaceutical composition according to claim 17, wherein the microcrystalline cellulose is present in the aqueous pharmaceutical composition at an amount of about 1.0% to about 10.0% by weight, preferably of about 1.4% to about 8.7% by weight, and most preferably at about 1.4%, 3.0%, 4.7%, 5.0%, 6.3%, 8.6%, or 8.7% by weight.

21. The aqueous pharmaceutical composition according to claim 17, wherein the microcrystalline cellulose has a nominal particle size of about 10 μm to about 50 μm, preferably of about 15 μm to about 25 μm, and most preferably of about 15 μm, 20 μm or 25 μm

22. The aqueous pharmaceutical composition according to claim 17, wherein the microcrystalline cellulose is selected from the group comprising Avicel® PH105, VivaPur® 105 and Emcocel® SP15.

23. The aqueous pharmaceutical composition according to claim 18, wherein the surfactant is present in the aqueous pharmaceutical composition at an amount of about 1.0% to about 5.0% by weight, preferably of about 1.4% to about 3.9% by weight, most preferably of about 1.4%, 2.9%, 3.0%, 3.3% or 3.9% by weight.

24. The aqueous pharmaceutical composition according to claim 18, wherein the surfactant is a polysorbate, preferably is Tween® 20 or Tween® 80.
